(19) United States
(12) Patent Application Publication
(10) Pub. No.: US 2018/0049734 A1
(43) Pub. Date: Feb. 22, 2018

(54) Method and Device for Securing Suture to an Anchor Body of a Suture Anchor

(71) Applicant: Conmed Corporation, Utica, NY (US)

(72) Inventor: Andrew Kam, Odessa, FL (US)

(73) Assignee: Conmed Corporation, Utica, NY (US)

(21) Appl. No.: 15/678,703

(22) Filed: Aug. 16, 2017

Related U.S. Application Data
(60) Provisional application No. 62/375,652, filed on Aug. 16, 2016.

(51) Int. Cl. A61B 17/04; A61B 17/06

(52) U.S. Cl. CPC A61B 17/0401; A61B 17/06166; A61B 2017/0409; A61B 2017/0414; A61B 2017/0464; A61B 2017/0441; A61B 2017/0445; A61B 2017/0459; A61B 2017/0451; A61B 2017/0406

(57) ABSTRACT

A suture anchor device includes: a suture anchor body; a filament portion connected to the suture anchor body, the filament portion comprising a first working end and a second working end extending from the suture anchor body, wherein: the second working end includes a hole positioned through the second working end; and the first working end is configured to be moveably positioned through the hole from a first side of the second working end to a second side of the second working end.

### CROSS-REFERENCE TO RELATED APPLICATIONS

The present application claims priority to and the benefit of U.S. Provisional Patent Application No. 62/375652, filed on Aug. 16, 2016, the entire contents of which are hereby incorporated by reference.

### FIELD OF THE INVENTION

The present disclosure relates generally to suture anchors. More particularly, the present disclosure relates to methods and devices for securing a suture to an anchor body of a suture anchor.

### BACKGROUND

Various conventional forms of anchors employed during surgical procedures are known. Anchors (such as cannulated knotless suture anchors disclosed in U.S. Pat. No. 8,409,252 assigned to the assignee hereof and incorporated by reference herein in its entirety) are commonly employed during surgical procedures to provide a reliable attachment location for sutures in or against a substrate, those attached sutures then being used to capture and retain other objects, such as soil tissue or bone. The substrate may be bony tissue or soft tissue such as ligaments, tendons, etc. In the case of bony tissue, suture anchor body portions of suture anchors are generally inserted into and retained in a pre-formed hole in the bone, so that suture extends from the anchor body out of the hole. In the case of soft tissue, suture anchors generally are placed on a side of the soft tissue such that suture extends through a hole in the tissue to extend beyond the soft tissue on a side opposite the anchor body.

Soft suture anchors have recently been developed from filaments of suture material which are retained within pre-formed bone holes by being deformable to increase their diameter to a size greater than that of the bone hole, to thereby reside within the cancellous bone and under the bone cortex. One such suture anchor is disclosed in U.S. Patent Publication No. 2012/0290004 assigned to the assignee hereof and incorporated by reference herein in its entirety. Since soft anchors are commonly made entirely of suture materials, they are sometimes called “all-suture” anchors, and generally include a fibrous construct anchor body portion (or fibrous, braided or woven fabric-type structure such as a flexible web, as described in U.S. Pat. No. 9,173,652) and a suture or filament portion. Methods and devices for inserting/deploying such all-suture anchors are known, examples of which are disclosed in U.S. Pat. No. 9,173,652.

As described in U.S. Pat. No. 8,409,252, for example, “non-soft,” “hard” or “rigid” suture anchors generally include a “hard” anchor body portion (that may or may not include inner and outer members) and a suture/filament portion. The anchor body of such suture anchors may be formed of a biocompatible and/or bioabsorbable material. These materials may be of such composition that they are reabsorbed by the body, e.g., during the healing process of the bone. Exemplary materials that are suitable for use in the inner and outer members include, but are not limited to, polyetheretherketone (“PEEK”), polylactic acid/beta-tricalcium phosphate (“PLA/Beta-TCP”) composites, ultra-high molecular weight polyethylene (“UHMWPE”), as well as other metallic, non-metallic, and polymeric materials.

However, the suture/filament portion of these conventional anchor devices can often slide through the anchor body/fibrous construct portion during deployment and/or fixation of the suture anchor to bone.

Accordingly, the inventor recognized that there is a need in the art for improved methods and devices for securing the suture/filament to the anchor body/fibrous construct of a suture anchor.

### DESCRIPTION OF THE RELATED ART SECTION DISCLAIMER

To the extent that specific patents/publications/products are discussed above in this Background Section or elsewhere in this Application, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/products are discussed above in this Background Section and/or throughout the application, the descriptions/disclosures of which are all hereby incorporated by reference into this document in their respective entirety(ies).

### SUMMARY OF THE INVENTION

The present disclosure is directed to inventive devices and methods for securing a suture/filament to an anchor body/fibrous construct of a suture anchor. A particular non-limiting goal of the utilization of the embodiments and implementations herein is to provide a device which is structured and/or configured to provide a secure construct by preventing the suture/filament from freely sliding through the anchor body/fibrous construct of a suture anchor in various medical procedures such as a double row rotator cuff repair of the shoulder (as should be understood by a person of skill in the art in conjunction with a review of this disclosure).

Generally, in one aspect, a suture anchor device includes: a suture anchor body; a filament portion connected to the suture anchor body, the filament portion comprising a first working end and a second working end extending from the suture anchor body, wherein: the second working end includes a hole positioned through the second working end; and the first working end is configured to be moveably positioned through the hole from a first side of the second working end to a second side of the second working end.

According to another aspect, a method for securing a filament portion to an anchor body of a suture anchor includes: providing a suture anchor comprising; a suture anchor body; a filament portion connected to the suture anchor body, the filament portion comprising a first working end and a second working end extending from the suture anchor body, wherein the second working end includes a hole positioned through the second working end, and positioning the first working end through the hole from a first side of the second working end to a second side of the second working end.

The details of one or more embodiments are described below and in the accompanying drawings. Other objects and advantages of the present invention will in part be obvious, and in part appear hereinafter.

### BRIEF DESCRIPTION OF THE DRAWINGS

The present invention will be more fully understood and appreciated by reading the following Detailed Description in conjunction with the accompanying drawings. The accompanying drawings illustrate only typical embodiments of the disclosed subject matter and are therefore not to be considered limiting of its scope, for the disclosed subject matter may admit to other equally effective embodiments.

Reference is now made briefly to the accompanying drawings, in which:

FIG. 1 is a schematic representation of an anchor body/fibrous construct of a suture anchor.

FIG. 2 is a schematic representation of a suture anchor.

FIG. 3 is a schematic representation of a suture anchor according to an embodiment.

FIG. 4 is a photographic representation of the suture anchor shown in FIG. 3 and positioned on an insertion/deployment device according to an embodiment.

FIG. 5 is a schematic representation of a suture anchor according to another embodiment.

Where applicable, like reference characters designate identical or corresponding components and units throughout the several views, which are not to scale unless otherwise indicated. Moreover, the embodiments disclosed herein may include elements that appear in one or more of the several views or in combinations of the several views.

### DETAILED DESCRIPTION OF EMBODIMENTS

Other patents/published patent applications may be discussed herein to illustrate some of the basic features of embodiments of the devices and methods that are part of this disclosure. However, the present disclosure is not limited to such basic features, which describes and illustrates features that are neither taught nor contemplated by such prior descriptions as will be set forth in detail in this section.

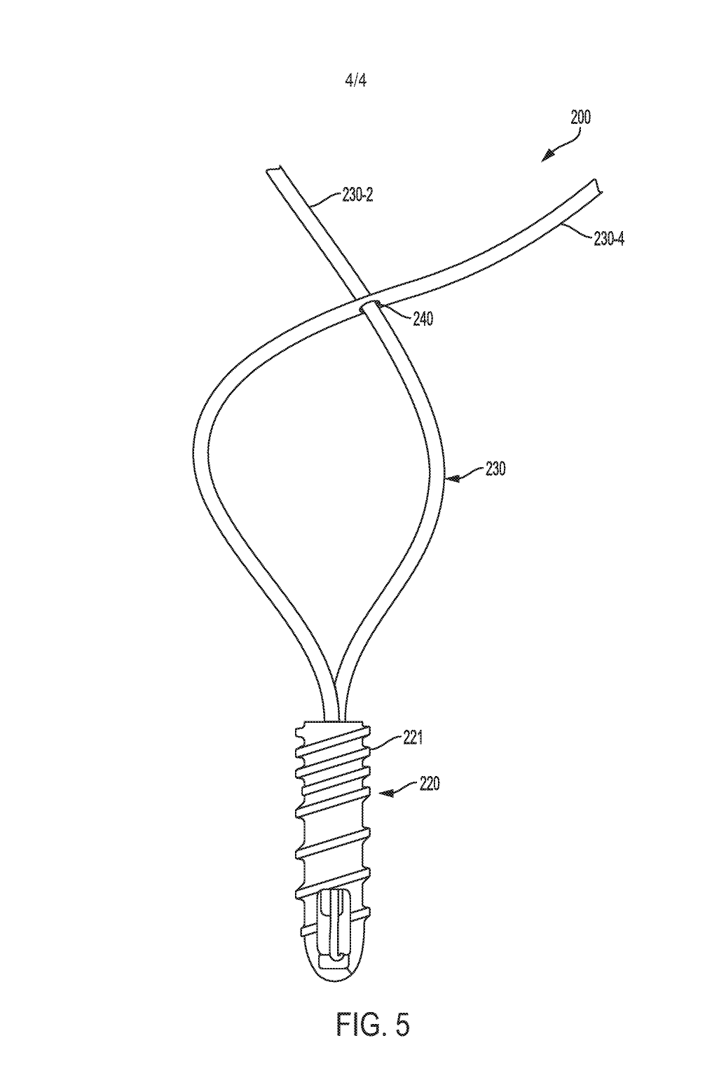

As described in U.S. 2012/0290004 (e.g., para. [0060] and shown in FIGS. 5 to 6; and also generally shown and described in U.S. Pat. No. 9,173,652), the soft anchor (or “all-suture” anchor) embodiments discussed herein can contain two sections: at least one filament, which is a suture to be anchored; and a fibrous construct (anchor body), which is to form a portion of the anchor that can increase in width, thickness and/or diameter and shrink in length as part of deployment. Even though it is the fibrous construct that increases in width, thickness and/or diameter at deployment, it should be understood that the filament also plays a role in the deployment of the anchor even though the filament may remain free (in some embodiments) to slide, and non-slidable in others (at least at a particular position or point in use) in relation to the fibrous construct. The filament helps to position, align and support the fibrous construct, such that if the filament were to be removed from the fibrous construct after deployment of the anchor, the fibrous construct may be free to spill (i.e., release), allowing the fibrous construct to collapse and shrink in size, allowing for easy and potentially undesirable) removal.

In other words, the fibrous construct has two primary functions. First, it becomes a base for the filament to slide within. Second, when compressed and/or pleated during deployment, the fibrous construct becomes more compact in one direction thereby expanding outwardly and increasing its overall diameter to create a retention capacity. This action of having the fibrous construct change in shape to increase its overall diameter is a useful characteristic which may be used advantageously to secure the anchor in a hole or against a bony or soft tissue. It is this combination of the expanding fibrous construct coupled with the filament remaining slidable (in some embodiments; and non-slidable in others, at least at a particular position or point in use) in relation to the fibrous construct that render embodiments of the present invention ideal for the reattachment of soft tissue to bone or soft tissue to soft tissue where it is desirable to pass sliding knots to secure a repair.

The term “standing end” may be used to refer to one or both of the ends of a filament that will ultimately be placed under load by a surgeon during surgery. In relation to at least one embodiment, the term “working end” is used to describe an end of a filament used to create a knot. This is the end that would be pulled to tighten the knot while pulling the standing end may cause to the knot to slide, as in the case of a slip knot.

Filaments and sutures, as the terms are used and described herein, includes braided (i.e., multi-filament) suture and monofilament suture as well as any other metallic or non-metallic filamentary or wire-like material suitable for performing the function of a suture. This material can include both absorbable and non-absorbable materials.

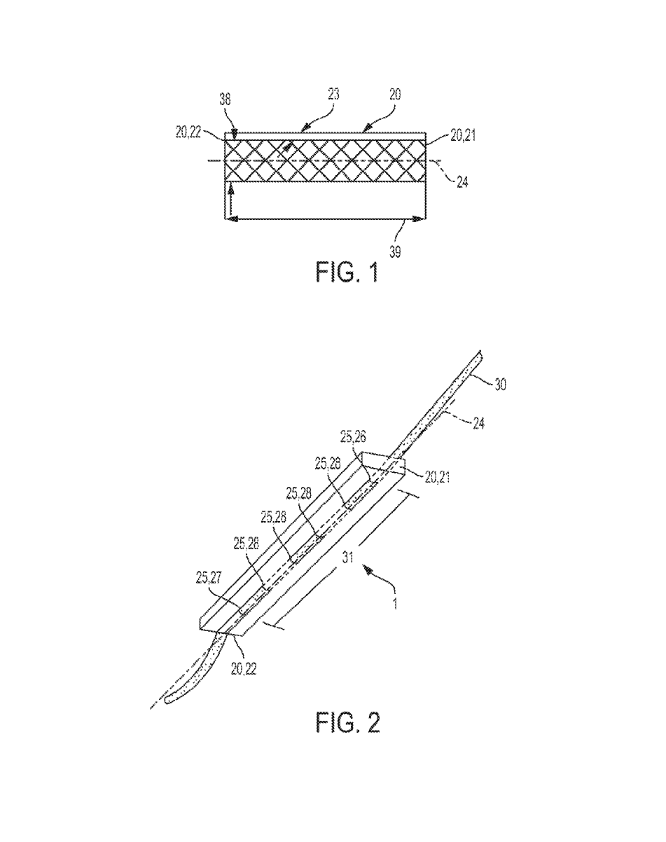

FIGS. 1-4 disclose a first embodiment of a suture anchor (soft/all-suture anchor). FIG. 5 discloses a second embodiment of a suture anchor (rigid anchor).

Referring to FIG. 1, in one embodiment, is a schematic representation of an anchor body/fibrous construct/suture tape 20 of soft/all-suture suture anchor 100. The fibrous construct 20 has a first end 20, 21 and a second end 20, 22 along with a mattress thickness 23, a width 38 and a length 39 along a longitudinal axis 24.

Turning to FIG. 2, in one embodiment, is a schematic representation of suture anchor 100. The suture anchor 100 can include a filament 30, which is passed through the fibrous construct 20 at passing locations 25. To help explain the function of the anchor 100, it may be helpful to define a first passing location 25, 26, which is a passing location 25 nearest the first end 21 of the fibrous construct 20. Similarly, a last passing location 25, 27, is a passing location 25 nearest the second end 22 of the fibrous construct 20. One or more intermediate passing locations 25, 28 are located in between the first passing location 25, 26 and last passing location 25, 27. Each passing location 25 is a location where the filament 30 passes through the mattress thickness 23 of the fibrous construct 20. In the present instance shown in FIG. 2, there are six passing locations 25. For reasons that will become more evident, as few as three passing locations 25 on a particular anchor 100 may function well. Similarly, more passing locations 25 may be provided, but there appears to be a reasonable limit where more passing locations provide no benefit or where additional passing locations actually hurt performance. It has been discovered that each additional passing location increases friction against the filament 30 thus reducing a surgeon's ability to slide the filament 30 in relation to the fibrous construct 20. Therefore, with any material change of the fibrous construct 20, some experimentation is expected to balance ability for the anchor to increase in size upon deployment against ability for the filament 30 to slide in relation to the fibrous construct 20. Lastly on this point, there may be even or odd numbers of passing locations 25.

All of the passing locations 25 in the anchor 100 of FIG. 2 occur along the longitudinal axis 24. Instead, the passing locations 25 may be offset a distance from the longitudinal axis 24 such that they are arranged parallel to the longitudinal axis 24. Similarly, the passing locations may be staggered such that one passing location is on one side of the longitudinal axis 24 while an adjacent passing location 25 is on an opposite side of the longitudinal axis 24.

Further descriptions of the general construction, material, methodology of use and functionality of certain aspect of the soft suture anchor and additional embodiments thereof are described in U.S. Patent Publication No. 2012/0290004 (e.g., multiple filaments) at FIGS. 5-15 and related disclosure.

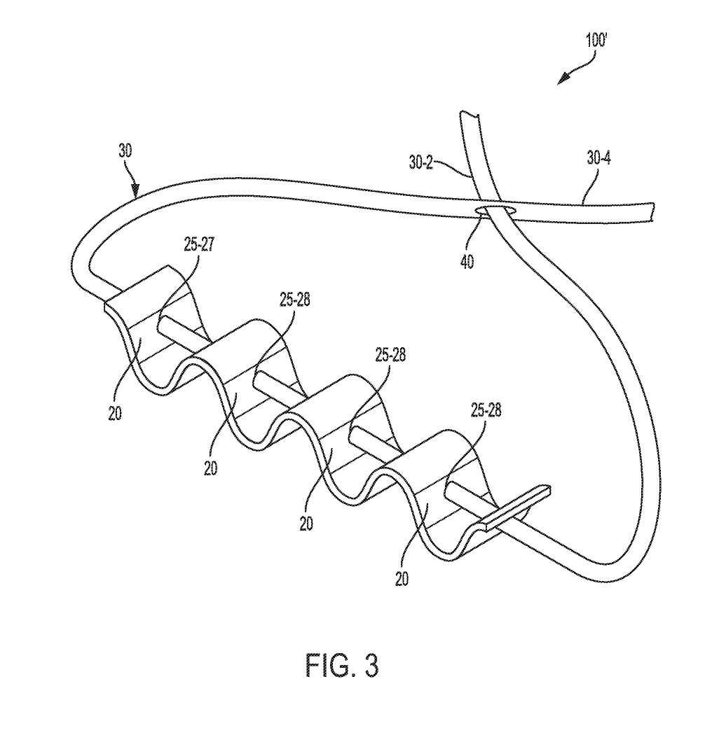

Referring to FIG. 3, in one embodiment, is a schematic representation of suture anchor 100′. This suture anchor 100′ embodiment includes similar elements to the suture anchor described and illustrated with respect to FIGS. 1-2. However, suture anchor 100′ also includes hole 40 positioned through at least one working end 30-4 of filament 30 at an angle that is perpendicular or transverse to the longitudinal axis of the at least one working end 30-4 (e.g., can be at any angle thereto). With the addition of hole 40 positioned through at least one working end 30-4, the other working end 30-2 of filament 30 can be passed through hole 40 as shown in FIG. 3. As such, the binding of the filament 30 to the fibrous construct 20 can be achieved and further enhanced by creating a “closed loop” around feature(s) of the fibrous construct 20 that form eyelet(s). The construction of this closed loop includes positioning a first working end 30-2 of filament 30 transversely through hole 40 of the second working end 30-4 (from a first side of the second working end 30-4 to the opposite side, and not back through to the first side) to create a loop with a first loop diameter. The constructed loop may be reduced to a smaller second loop diameter by placing tension on the first working end 30-2 in the direction away from hole 40 until a limit is reached (based on tactile or visual feedback) around the portion(s) of the fibrous construct 20 to which the loop is created around (which can vary based on the particular application of anchor 100′, as should be understood by a person of skill in the art in conjunction with a review of this disclosure).

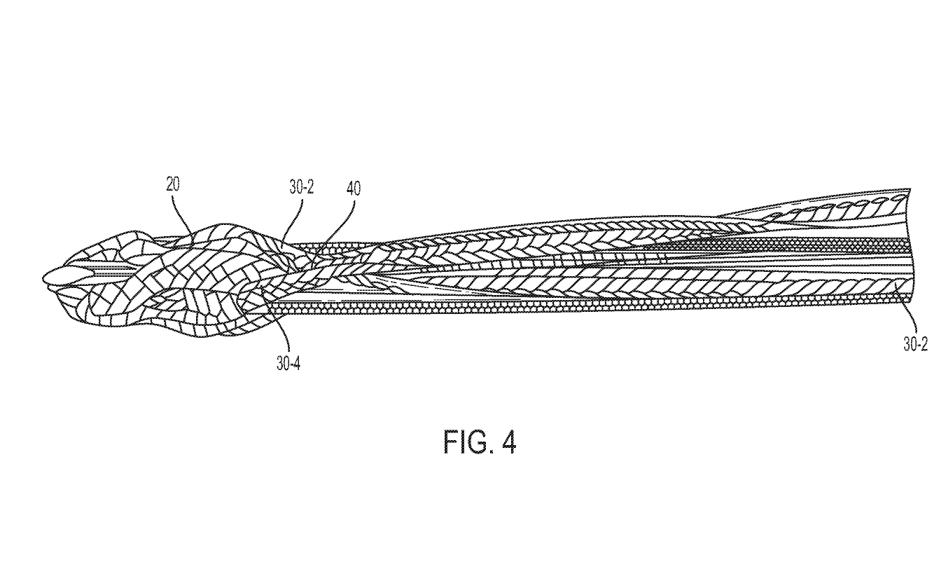

Referring to FIG. 4, in one embodiment, is a photograph of suture anchor 100′ positioned on an inserter or insertion/deployment device (such as a device shown and described in U.S. Pat. No. 9,173,652). The “loop” formed by fibrous construct 20 and filament 30 referenced above has been reduced in size after the first working end 30-2 was positioned through the hole 40 of the second working end 30-4 (from a first side to a second side), and the distal end of the first working end 30-2 was pulled tight to the smaller second loop diameter providing a secure construct by preventing the suture/filament from freely sliding through the fibrous construct.

Referring to FIG. 5, in another embodiment, is a schematic representation of rigid suture anchor 200. The same concept of the creation of the closed loop and functionality related to the secure construct discussed above with respect to the fibrous construct can be applied to the rigid anchor embodiment. The suture anchor 200 can include a filament 230, which is connected to and extends from the proximal end of an anchor body 220. The anchor body can include helical portions 221 of various spacing along the shaft of the anchor body 220. Similar to the embodiment shown and described with respect to FIGS. 3-4, suture anchor 200 also includes hole 240 positioned through at least one working end 230-4 of filament 230 at an angle that is perpendicular or transverse to the longitudinal axis of the at least one working end 230-4 (e.g., can be at any angle to). With the addition of hole 240 positioned through at least one working end 230-4, the other working end 230-2 of filament 230 can be passed through hole 240 as shown in FIG. 5. As such, the binding of the filament 230 to the anchor body 220 can be achieved by creating a “closed loop” around at least a portion of or adjacent to the anchor body 220. The construction of this closed loop includes positioning a first working end 230-2 of filament 230 transversely through hole 240 of the second working end 230-4 (from a first side of the second working end 230-4 to the opposite side, and not back through to the first side). The constructed loop may be reduced to a smaller second loop diameter by placing tension on the first working end 230-2 in the direction away from hole 240 until its limit is reached (based on tactile or visual feedback, and which can vary based on the particular application of anchor 100′, as should be understood by a person of skill in the art in conjunction with a review of this disclosure). Similar to the fibrous construct embodiment discussed above, this configuration provides a secure construct by preventing the suture/filament from freely sliding through the rigid anchor body 220.

Embodiments of the present disclosure contemplate more than one hole positioned through one or more working ends of at least one or more filaments, so that the other working end (or working ends of other filaments) can be passed therethrough.

While embodiments of the present invention has been particularly shown and described with reference to certain exemplary embodiments, it will be understood by one skilled in the art that various changes in detail may be effected therein without departing from the spirit and scope of the invention as defined by claims that can be supported by the written description and drawings. Further, where exemplary embodiments are described with reference to a certain number of elements it will be understood that the exemplary embodiments can be practiced utilizing either less than or more than the certain number of elements.

What is claimed is:

1. A suture anchor device comprising:
a suture anchor body;
a filament connected to the suture anchor body, the filament comprising a first working end and a second working end extending from the suture anchor body, wherein:
the second working end includes a hole positioned through the second working end; and
the first working end is configured to be moveably positioned through the hole from a first side of the second working end to a second side of the second working end.

2. The suture anchor of claim 1, further comprising a loop formed by the first working end positioned through the hole from the first side of the second working end to the second side of the second working end.

3. The suture anchor of claim 2, wherein in an undeployed state the loop has a first diameter, and in a deployed state the loop has a second diameter, the second diameter being less than the first diameter.

4. The suture anchor of claim 3, wherein the suture anchor body is a rigid anchor.

5. The suture anchor of claim 3, wherein the suture anchor body is a fibrous construct.

6. The suture anchor of claim 5, wherein the filament comprises an intermediate portion passing through the construct in at least three passing locations comprising a first passing location nearest a first end of the construct, a last passing location nearest a second end of the construct, and at least one intermediate passing location between said first passing location and said last passing location.

7. The suture anchor of claim 5, wherein in the undeployed state, the fibrous construct has a first fibrous construct width or thickness and a first fibrous construct length, and each filament extends a first filament length between said first passing location and said last passing location, and wherein in a deployed state, the fibrous construct has a second fibrous construct width or thickness and a second fibrous construct length, and each filament extends a second filament length between said first passing location and said last passing location, the second fibrous construct width or thickness being greater than the first fibrous construct width or thickness, the second fibrous construct length being shorter than the first fibrous construct length, and the second filament length being shorter than said first filament length.

8. The anchor of claim 6 wherein the fibrous construct is a ribbon having a longitudinal axis extending between the first end and the second end, a length along the longitudinal axis, a mattress thickness and a width.

9. The anchor of claim 8 wherein the passing locations are arranged along or parallel to said longitudinal axis.

10. The anchor of claim 8 wherein said passing locations are arranged in a staggered fashion crossing the longitudinal axis.

11. A suture anchor device deployment system comprising:
a suture anchor insertion device comprising a proximal end and a distal end;
a suture anchor body positioned on the distal end of the insertion device;
a filament connected to the suture anchor body, the filament comprising a first working end and a second working end extending from the suture anchor body, wherein:
the second working end includes a hole positioned through the second working end; and
the first working end is configured to be moveably positioned through the hole from a first side of the second working end to a second side of the second working end.

12. The system of claim 11, further comprising a loop formed by the first working end positioned through the hole from the first side of the second working end to the second side of the second working end.

13. The system of claim 12, wherein in an undeployed state the loop has a first diameter, and in a deployed state the loop has a second diameter, the second diameter being less than the first diameter.

14. A method for securing a filament to an anchor body of a suture anchor comprising:
providing a suture anchor comprising:
a suture anchor body;
a filament connected to the suture anchor body, the filament comprising a first working end and a second working end extending from the suture anchor body, wherein the second working end includes a hole positioned through the second working end; and
positioning the first working end through the hole from a first side of the second working end to a second side of the second working end to form a loop.

15. The method of claim 14, further comprising the step of positioning the suture anchor body on a distal end of a suture anchor insertion device.

16. The method of claim 15, further comprising the step of positioning the distal end of the suture anchor insertion device into a pre-formed bone hole.

17. The method of claim 16, further comprising the step of decreasing the diameter of the loop by pulling on the first working end away from the hole.

18. The method of claim 17, wherein the suture anchor body is a rigid anchor.

19. The method of claim 17, wherein the suture anchor body is a fibrous construct.

20. The method of claim 19, further comprising the step of increasing a width or thickness of the fibrous construct and decreasing a length of the fibrous construct by pulling on at least one of the first working end and the second working end.
